(19) United States
(12) Patent Application Publication
(10) Pub. No.: US 2025/0195228 A1
(43) Pub. Date: Jun. 19, 2025

(54) MEDICAL IMPLANT, IN PARTICULAR CONE AUGMENT

(71) Applicant: Waldemar Link GmbH & Co. KG, Hamburg (DE)

(72) Inventor: Helmut D. LINK, Hamburg (DE)

(73) Assignee: Waldemar Link GmbH & Co. KG, Hamburg (DE)

(21) Appl. No.: 18/848,580

(22) PCT Filed: Aug. 30, 2023
(86) PCT No.: PCT/EP2023/073833

§ 371 Date: Sep. 19, 2024

Related U.S. Application Data
(60) Provisional application No. 63/373,863, filed on Aug. 30, 2022.

(51) Int. Cl. A61F 2/30

(52) U.S. Cl. CPC A61F 2/30734; A61F 2/30771; A61F 2002/30161; A61F 2002/30518; A61F 2002/30593; A61F 2002/30594; A61F 2002/30616; A61F 2002/30624; A61F 2002/30736; A61F 2002/3092; A61F 2002/3093

(57) ABSTRACT

Medical implant, in particular an augment for a joint endoprosthesis, comprising a sleeve-shaped hollow body having a wall surrounding a channel in a circumferential direction. The channel extends in a longitudinal direction from a bottom to a top of the hollow body. The wall comprises at least one living hinge oriented in the longitudinal direction. The living hinge is configured for compressing the channel. The living hinge being formed by a bent slit that extends as a slit at least in a vertical and in a horizontal direction. As opposed to an ordinary linear slit, the bent, i.e., non-linear slit extends thus as a slit in two dimensions, horizontal and vertical. Thereby an increased elasticity can be realized, achieving a medical implant which has a better adjustable elasticity.

### CROSS REFERENCE TO RELATED APPLICATIONS

This application claims priority to U.S. Provisional Application No. 63/373,863 filed Aug. 30, 2022, the entire contents of which are incorporated by reference herein.

### TECHNICAL FIELD

The present disclosure relates generally to a medical implant, and more particularly to implantable structures such as cone augments related to bending joints for bones, such as the tibial, femoral, or humeral bones.

### BACKGROUND

Patients who suffer from injury or illness related to musculoskeletal or orthopedic issues often require endoprostheses, or artificial implants, to mimic and connect to bones and joints. Implants must be manufactured with material and designs that provide strength comparable to an original bone or join. Additionally, many bone implants or prostheses that interface with a joint require some degree of flexibility to allow for maximal movement, and ease of installation.

Long bone implants may be designed to fit within a cone augment, which provides mechanic support to the bone implant. Such augments themselves are implants and are configured to have dimensions in accordance with the bone or implant that will be placed therein. Such cones must not only be sufficiently strong to support the implant or bone it interfaces with, but also provide an appropriate amount of flexibility for ease of install.

Such “cones” which are to be understood as augments being typically (but not necessarily) cone-shaped provide support for larger bones, such as a tibia or femur bone, are relatively large. In certain designs of such cones (such as disclosed in EP 3 319 558 A1), a bending joint is incorporated within the cone, where the outer wall of the cone includes a portion that is non-contiguous and separated, allowing for some degree of expansion/contraction. Cones designed for smaller bones, however, have smaller diameter, which increase the stiffness of the cone's outer wall when manufactured from the same material and the same wall thickness as the larger cones. This increase in stiffness limits the elasticity of the cone and restricts its usage.

Further, cone augments are known having a single narrow longitudinal slit at two or three locations around their wall (EP 4 197 496 A1) or at more locations (TrabecuLink Cones marketed by Waldemar Link GmbH & Co KG, Hamburg, DE).

Thus it is an object of the invention to provide an improved design is desired allowing for providing more elasticity of the subject augment implants.

### BRIEF SUMMARY OF THE DISCLOSURE

The solution according to the invention resides in the features of the independent claims. Advantageous embodiments are the subject of the dependent claims.

In a medical implant, in particular a (cone) augment for a joint endoprostheses, comprising a sleeve-shaped hollow body having a wall surrounding a channel in a circumferential direction, the channel extending in a longitudinal direction from a bottom to a top of the hollow body, the wall comprising at least one living hinge oriented in the longitudinal direction, said living hinge being configured for compressing the channel and being formed by at least one elongated surface opening, according to the invention said elongated surface opening is a slit that is bent and extends as a slit at least in a vertical and in a horizontal direction.

The core aspect of the invention is to provide the medical implant with a bent, i.e. non-linear, slit that is extending in both, vertical and horizontal, directions. (The directional terms refer to an orientation of the medical implant wherein the longitudinal direction of the channel is essentially upright, and the horizontal direction is essentially along a circumference of the wall, as detailed below.) Thereby the bent slit extends as a slit in two dimensions, as opposed to an ordinary linear slit extending only in one.

Thereby the effect can be achieved, that by having the slit extending in two dimensions a much more effective increase of elasticity of the wall can be realized, thereby achieving a medical implant, in particular cone augment, which has more elasticity to become more adaptable to dimensional differences of the receiving bone. Particularly, by utilizing the horizontal (along the circumference) portion of the bent slit a valuable additional means of achieving a higher elasticity is provided. By employing the concept of a two-dimensional bent slit according to the invention, elasticity as well as the range for adjusting elasticity can be increased.

The key element of the invention, the bent slit being extended in both, vertical as well as horizontal, direction, has further a significant advantage in that dual adjustability is provided. Increasing the vertical extension of the bent allows for increasing of the portion of the wall which is passive and does not participate in providing elastic counter-force to a compressional force. On the other hand, by increasing the horizontal extension of the bent slit allows for an adjustment of the elasticity, i.e. longer horizontal extension provide for an increased softness with respect to the compressional force. Thereby, two parameters, namely size as well as stiffness/softness of the effective elastic joint can be determined by the extension of the bent slit in vertical and horizontal direction, respective. This provides for a much-improved adjustability. This further contributes to enabling even small sizes of a medical implant to feature elastic characteristics like those of the larger sizes.

As a result, owing to the bent slits the invention provides a medical implant having a much improved elasticity, thereby allowing a greater compression of the channel. This enables a better fitment to a bone cavity, and ensures that even smaller sizes of the medical implant (smaller diameter/width and/or lesser height) can be provided with a greater elasticity. Usability and versatility of the implants can be improved thereby.

The longitudinal direction extends from a bottom to a top of the hollow body. It is substantially perpendicular to the circumferential direction of the wall. A radial direction is any direction orthogonal to the longitudinal direction (like spokes of a wheel whose rotational axis coincides with the longitudinal direction).

A horizontal direction, in particular of a slit or a portion/section thereof is understood to be essentially parallel to the circumferential direction of the wall. A vertical direction, in particular of a slit or a portion/section thereof, is understood to be essentially perpendicular to the horizontal direction and in plane with the respective portion of the wall.

Extending at least in the horizontal direction” may be achieved also by a section of the slit having an oblique (with respect to the vertical) orientation. This allows an additional range of beneficial shapes for the bent slits that facilitate achieving the desired increase of elasticity. Further, an improved manufacturing can be achieved thereby, in particular by 3D-printing manufacturing, if the oblique section is oriented at an angle of at least 45°, preferably 50° to 65°, further preferably about 55°, yet further preferably less than 75° to the horizontal direction, however.

The slits can be sharply bent so as to be formed by at least two connected sections which are angled to each other. However, a sharp bent as a transition between such sections is not necessary for being a bent slit as claimed, the bent slit formation may also be achieved by a curved slit. Such a soft bended, curved transition from vertical to horizontal may be beneficial in order to improve long-term stability and to reduce any risk of material fatigue as opposed to angled slits.

Preferably, a groove is provided for the living hinge, said groove running in a longitudinal direction at an inner and/or outer face of the wall. Owing to the groove an effective thickness of the wall is reduced at a position where the living hinge is located, thereby contributing to an increased elasticity of the living hinge. In most cases, the bent slit will be arranged exclusively at a ground of the groove, but this is not a necessity. Further, it is preferred that the groove runs along the entire height of the hollow body, i.e. from the bottom to the top of the wall.

Advantageously, a depth of the bent slit has a depth of at least a third, preferably at least a half, of a thickness of wall. Such a depth may suffice to achieve a significant elastic characteristic by means of the bent slit. Increased depth generally provides for an increased elasticity (all else being equal).

It is especially preferred to configure the bent slit such as to be a through opening. By virtue of the through opening, certain portions of the wall are decoupled from providing counter-force upon compression, thereby further increasing elasticity to make the living hinge more soft. Advantageously, the through opening formed by the bent slit has a width that is so small to block passage of bone cement, preferably a width being 0.5 mm or less, further preferably 0.4 mm or less. Thereby a cement sealing is formed. Bone cement present in the channel can therefore by blocked from outflowing to the exterior of the sleeve-shaped hollow body. Any risk of clogging porous structures that may be provided on an outer face of the wall can thereby be effectively avoided.

Preferably, the bent slit is a framed slit. Thereby, it is completely surrounded by material of the wall, in other words the bent slit is closed and not open to a side.

A variety of geometries can be employed for the bent slits. Some geometries may be preferable. In the following some non-limiting examples for preferable embodiments will be provided. As a first preferable embodiment, the bent slit may be formed as a chevron having at least two sections being oriented obliquely to the vertical (longitudinal) direction. This has the benefit of a rather simple structure that notwithstanding its simplicity achieves well the desired effect on elasticity. A non-limiting example is a chevron having a basic orthogonal tip angle between its sections, however said angle could alternatively be blunt or acute.

As a second preferable embodiment, the bent slit may be formed by a vertical base section and at least one horizontal section positioned at an end portion of the vertical base section, said at least one horizontal section being preferably oriented oblique or orthogonal to the vertical. Particularly, two horizontal sections may be provided, preferably one at or near either end of the vertical base section. This has the benefit of forming a U-Like shape of the bent slit, thereby circumscribing a flap like shape which is beneficial for achieving a softer and well controlled elasticity. The horizontal sections may be arranged in parallel, even if they are at an oblique angle to the vertical base section. Thereby, for the same living hinge a series (as seen in the vertical direction) of such bent slits can be provided with a minimum of separating space between them. A non-Limiting example for a non-orthogonal variant features an angle of (90°−55°=) 35° between a vertical and an oblique section.

A series of bent slits is understood to be a living hinge having a plurality of bent slits being placed one after the other in the vertical direction but having essentially the same (horizontal) position in the circumferential direction; however, a staggered positioning of the bent slits of a series is possible. Preferably, the series comprises more than two bent slits thereby effectively providing bent slits over a majority of the distance from the bottom to the top of the wall. A particular benefit of such a stacking of bent slits is, as opposed to a long slit of comparable combined length, that strip-like lands of solid wall material will remain between the stacked bent slits, thereby increasing stability of the medical implant.

Further, as a third preferable embodiment a combination of the first and second may be realized, wherein a chevron is provided at or near either end of the vertical base section. Thereby a geometry Like that of a feathered arrow can be achieved, which is highly effective for forming a dense series of such (multiple) bent slits with minimum spacing between them.

As a preferred variant relating to all embodiments, a transition between sections can be sharp angled or curved. Either type of transition is acceptable to the invention. A sharp angled transition has the benefit of being more compact and requiring less space, and-depending of manufacturing-being easier to form. On the other hand, a curved transition, e.g. a 90° arc of a circle, has the benefit of a smoother transition and eliminating any risk of an adverse notch effect. A particularly beneficial embodiment is a continuously curved slit, like a semi-circular arch.

Advantageously, a bending strip is formed between two neighbouring bent slits of a series of bent slits. Thereby, the bending strip has slits at either of its lateral sides, whereas at its front and back portion it is in direct continuity with the adjacent wall. Being arranged between the (bent) slits, the bending strip is thus essentially the device which delivers a majority of the counterforce to elastic compression of the cone augment. In other words, the bending acts like a leaf sprint in order to produce the counterforce which opposes the compression, and therefore defines the elasticity of the cone augment. The same applies with respect to interspace between a peripheral bent slit and the adjacent top/bottom of the wall.

If the bent slits of a living hinge are placed rather close to each other, the resulting bending strip will be rather narrow. Such a narrow bending strip acts like a narrow leaf spring (as opposed to a wide leaf spring) and produces a relatively weak counterforce, therefore resulting in a cone augment of rather high elasticity. Conversely, if the bent slits of the living hinge are placed with a bigger spacing from each other, then the resulting bending strips is wider and it will produce, like a wide leaf spring, a stronger counterforce, therefore resulting in a cone augment of lesser elasticity (i.e. harder to compress). Accordingly, a predetermined elasticity of the living hinge—and as a consequence of the cone augment—can be achieved easily by dimensioning a width of the bending strip and placing the bent slits of each series accordingly.

Preferably, a plurality of oblique bending strips are formed between three or more of the bent slits having oblique sections, said bending strips being oriented obliquely in opposite directions. Such an opposite direction arrangement of the obliquely oriented bending strips has the advantage that upon compression any forces pointing in the longitudinal direction (upward or downward if the cone augment stands on its bottom) cancel each other out, thereby achieving a smooth compression without any risk of other unwanted movements as adverse side-effects.

Advantageously, a plurality of the living hinges may be provided along the wall. Preferably, said hinges are spaced equidistant and/or equiangular along the circumferential direction of the wall. Such plurality allows for an even softer elasticity and further increases compressibility. Moreover, a plurality allows for a more even distribution of the resulting reduction of the channel width and is therefore beneficial for maintaining the general shape of the cone augment even if heavily compressed. It the cone augment has an axis of symmetry, then it would be further beneficial to arrange said plurality also symmetrically to said axis.

Preferably, the bent slits within any of the living hinges can feature a combination of various different geometries of the bent slits. This also applies to a single living hinge. Further, if there are a plurality of living hinges the geometries of the bent slits may differ between those living hinges. So, different geometries of the living hinges may be present inter and intra the living hinge(s).

Advantageously, the hollow body comprises a compensator element configured for adjusting a circumference of the wall in a compressed state. The compensator element allows a degree of freedom for absorbing a reduction of the circumference which will be realized by a compressive force acting on the exterior of the wall with its elastic living hinges.

Preferably, the compensator element is configured as a longitudinal wall void extending from the bottom to the top of the wall to form a first and second edge of the wall separated by the width of said elongated longitudinal opening. Thereby the wall will be non-contiguous. Said longitudinal wall void is preferably configured like a slot, and it may be a straight slot or serrated, wherein teeth at the edges forming the serration are preferably pointed, e.g. triangular, or blunt, e.g. rectangular.

Further preferably, the first and second edges are provided with over-lapping tongue-like extensions arranged in a sliding relationship. Owing to the sliding relationship, the tongues act as a sealing against cement flow, thereby essentially maintaining the bulkhead functionality even in the area of the compensator element. Further, the sliding relationship allows a variance in width and thereby the reduction of the circumference. The sliding relationship may comprise direct contact or just close proximity such as to form a gap small enough to block flow of bone cement.

In a preferred embodiment, the wall has an inner face to the channel and an outer face on the opposite side of the wall facing to the exterior, wherein the outer face at least partially comprises porous structure configured for bone ingrowth. By virtue of the porous structure, ingrowth of bony material is being promoted, thereby achieving an improved long-term stability. The inner face, on the other hand, is preferably solid. The solid face acts as a kind of bulkhead avoiding that any cement flows through the wall. Without such a bulkhead there would be the risk that the cement flows from the interior through the wall and fills the porous structure, thereby blocking ingrowth of bony material which would lead to the unwanted consequence of an unsatisfactory fixation of the medical implant to the surrounding bone.

Advantageously, the wall comprises pockets in which the porous structure is affixed. Thereby well-defined zones can be formed wherein the porous structure shall be present and where ingrowth of bone material shall happen. The porous structure is preferably configured to be osteoconductive. The remaining portions of the wall may be solid and therefore mechanically more robust and capable of bearing higher loads. Preferably, the pockets have such a depth that the porous structure has at least one, preferably two or more layers of pores for proper ingrowth of the bony material. The pores are preferably interconnected which allows for a stronger fixation by ingrown bony material.

The medical implant is preferably made of biocompatible material, preferably metal or zirconium, further preferably a material selected from a group comprising pure titanium like e.g. titanium grade 2, a titanium alloy, like e.g. Ti6Al4V, tantalum, cobalt chromium and stainless steel. These are biocompatible materials which provide sufficient strength and long-term stability for the medical implant.

It is particularly preferred to form the medical implant as a unitary device, preferably including the porous structure. Thereby a smooth surface without protruberances can be achieved, with the benefit of keeping low irritation of surrounding bone and tissue material.

In a particularly advantageous embodiment that may deserve independent protection, the medical device is formed by additive manufacturing, preferably by 3D printing. Thereby even embodiments with a plurality of living hinges having complex shaped bent slits can be efficiently manufactured including any porous structure. As a result, reliable and efficient manufacturing can be performed in an economical manner.

Preferably the medical implant is configured as an augment device for a joint endoprosthesis, wherein the channel is configured for receiving a stem of said joint endoprosthesis. For proper anchoring of the joint endoprosthesis in the adjacent bone, typically a long bone, a proper seating at the bone is necessary. If the bone is defective, either due to illness or due to removal of fractured or otherwise defective bone material, then an augment is beneficial for providing proper support for the endoprosthesis including its stem. The medical implant as an augment according to the present invention is particularly suited for usage at sites having highly defective bone material since a rather large compression of the medical implant for better fitment can be achieved owing to the superior adjustment of elasticity enabled by the present invention.

Specifically, the medical implant according to the present invention may be used as an implant cone (cone augment) for an articulated joint endoprosthesis, preferably as a tibial, femoral, or humeral implant cone, or as a dental cone.

The invention further relates to a set of medical implants as described herein, the set comprising a plurality of such medical implants, in particular cone augments, with at least one larger and one smaller medical implant. By virtue of such a set, a variety of different bone sizes can be provided with a properly dimensioned medical implant. Preferably, within said set the smaller medical implant features a bending strip having a higher length/width ratio than that of the bending strip the larger medical implant, in particular cone augment. Such a higher length/width ration could be achieved by the bending strip having a greater length or a smaller width, or both. Thereby it can achieved that a medical implant of the smaller size can have the same elasticity compared to that of the larger medical implant, which is often desirable (or even a higher elasticity if so wished).

The terms “larger” and “smaller” refer to different sizes of the medical implant, wherein different sizes are understood such that the medical implants differ in diameter/width, and/or height, and/or dimension of the free space in the interior of the wall forming the channel.

The invention is explained in more detail by way of examples in conjunction with the accompanying drawing showing advantageous embodiments. In the drawing:

### BRIEF DESCRIPTION OF THE DRAWINGS

FIG. 1a, b are a horizontal cross-sectional view of first and second embodiments of the invention;

FIG. 2a, b are an elevated lateral view of said first and second embodiments;

FIG. 3a, b are a perspective view of a vertical cross-section of said first and second embodiments;

FIG. 4a, b are lateral side views of said first and second embodiments;

FIG. 5 is a detail view of a portion marked “V” in FIG. 4 showing an horizontal extension of bent slits;

FIG. 6a, b, c are lateral side views of a third fourth and fifth embodiment of the invention;

FIG. 7a, b, c are schematic views of basic geometries of bent slits;

FIG. 8a, b, c are schematic lateral views showing bending strips formed between bent slits;

FIG. 9 is a schematic view showing an obliquely angled portion of a bent slit;

FIG. 10 is a schematic view showing an exemplary embodiment of the invention in situ;

FIG. 11a, b, care perspective views of a sixth embodiment in different sizes;

FIG. 12a, b show details of bending strip formation of different sizes of a seventh embodiment;

FIG. 13a, b show top views of the different sizes of said seventh embodiment;

FIG. 14a-d are schematic lateral views of different embodiments of series of bent slits;

FIG. 15 are schematic frontal views of the embodiments of FIG. 14a, b;

FIG. 16 is a perspective view of a known augment; and

FIG. 17a, b are schematical views showing length of lever arms determining elasticity at known augments.

### DETAILED DESCRIPTION

To facilitate the understanding of this disclosure a number of terms of in quotation marks are defined below. It is noted that the drawings of the present application are provided for illustrative purposes only and, as such, the drawings are not drawn to scale. It is also noted that like and corresponding elements are referred to by like reference numerals.

In the following description, numerous specific details are set forth, such as particular structures, components, materials, dimensions, processing steps and techniques, in order to provide an understanding of the various embodiments of the present application. However, it will be appreciated by one of ordinary skill in the art that various embodiments of the present application may be practiced without these specific details. In other instances, well-known structures or processing steps have not been described in detail in order to avoid obscuring the present application.

It will be understood that when an element as a layer, region or substrate is referred to as being “on” or “over” another element, it can be directly on the other element or intervening elements may also be present. In contrast, when an element is referred to as being “directly on” or “directly over” another element, there are no intervening elements present. It will also be understood that when element is referred to as being “beneath” or “under” another element, it can be directly beneath or under the other element, or intervening elements ay be present. In contrast, when an element is referred to as being “directly beneath” or “directly under” another element, there are no intervening elements present.

As used herein, the term “substantially” or “substantial”, is equally applicable when used in a negative connotation to refer to the complete or near complete lack of an action, characteristic, property, state, structure, item, or result. For example, a surface that is “substantially” flat would either be completely at, or so nearly flat that the effect would be the same as if it were completely flat.

As used herein, terms defined in the singular are intended to include those terms defined in the plural and vice versa.

As used in this specification and its appended claims, terms such as “a”, “an” and “the” are not intended to refer to only a singular entity, but include the general class of which a specific example may be used for illustration, unless the context dictates otherwise. The terminology herein is used to describe specific embodiments of the disclosure, but their usage does not delimit the disclosure, except as outlined in the claims.

Unless otherwise indicated, all numbers expressing quantities of ingredients, properties such as molecular weights, reaction conditions, and so forth as used in the specification and claims are to be understood as being modified in all instances by the term “about.” Accordingly, unless indicated to the contrary, the numerical parameters in the specification and claims are approximations that may vary depending upon the desired properties sought to be obtained by the present disclosure. At the very least, and without Limiting the application of the doctrine of equivalents to the scope of the claims, each numerical parameter should at least be construed in light of the number of reported significant digits and by applying ordinary rounding techniques. Notwithstanding that the numerical ranges and parameters describing the broad scope of the disclosure are approximations, the numerical values in the specific examples are reported as precisely as possible. Any numerical value, however, inherently contains standard deviations that necessarily result from the errors found in the numerical value's testing measurements.

Thus, reference herein to any numerical range expressly includes each numerical value (including fractional numbers and whole numbers) encompassed by that range. To illustrate, reference herein to a range of “at least 50” or “at least about 50” includes whole numbers of 50, 51, 52, 53, 54, 55, 56, 57, 58, 59, 60, etc., and fractional numbers 50.1, 50.2 50.3, 50.4, 50.5, 50.6, 50.7, 50.8, 50.9, etc. In a further illustration, reference herein to a range of “less than 50” or “less than about 50” includes whole numbers 49, 48, 47, 46, 45, 44, 43, 42, 41, 40, etc., and fractional numbers 49.9, 49.8, 49.7, 49.6, 49.5, 49.4, 49.3, 49.2, 49.1, 49.0, etc. In yet another illustration, reference herein to a range of from “5 to 10” includes whole numbers of 5, 6, 7, 8, 9, and 10, and fractional numbers 5.1, 5.2, 5.3, 5,4, 5,5, 5.6, 5.7, 5.8, 5.9, etc.

In the discussion and claims herein, the tern “about” indicates that the value listed may be somewhat altered, as long as the alteration does not result in nonconformance of the process or structure to the illustrated embodiment. For example, for some elements the term “about” can refer to a variation of ±0.1%, for other elements, the term “about” can refer to a variation of ±1% or ±10%, or any point therein.

Any portion, or all of, of any of the implantable structures disclosed herein can be formed wholly or partially in any suitable way, such as through any suitable additive manufacturing (AM) and/or 3D-printing technology. For example, the hollow body 10 of any of the implantable structures can be formed according to a suitable electron beam melting (EBM) process and/or a suitable selective laser melting (SLM) process.

EBM is an additive process for manufacturing and may produce solid or porous material. A powder of the desired material is provided in the desired granulometry. By the EBM process the powder is deposited in successive layers and subsequently made to melt, by virtue of an electron beam (EB), at desired positions according to a preceding modelling step to form a coherent, solid body, such as any portion of, or the entirety of, any implantable structure disclosed herein.—SLM is essentially the same process, just employing a different energy source to create the energy beam, which is a selective laser beam (SL) as opposed to an electron beam (EB).

As one example of this AM technology, the hollow body of a medical implant, e.g. a cone augment 1, can be additively manufactured from Ti-6Al-4V extra low interstitial (ELI) powder in an electron beam machine (Arcam EBM Q10 plus).

The invention will be illustrated using an embodiment of the medical implant which is a cone augment. It may be of a generally cylindrical or conical form.

The cone augment 1 is generally configured as a sleeve-like hollow body 10 having a wall 2 surrounding a channel 11 which runs through the hollow body 10 from a bottom 13 to a top 12 of the hollow body 10. The main axis of the channel 11 defines a longitudinal direction 15. The distance between the bottom 13 and the top 12 defines a height 14 of the augment 1.

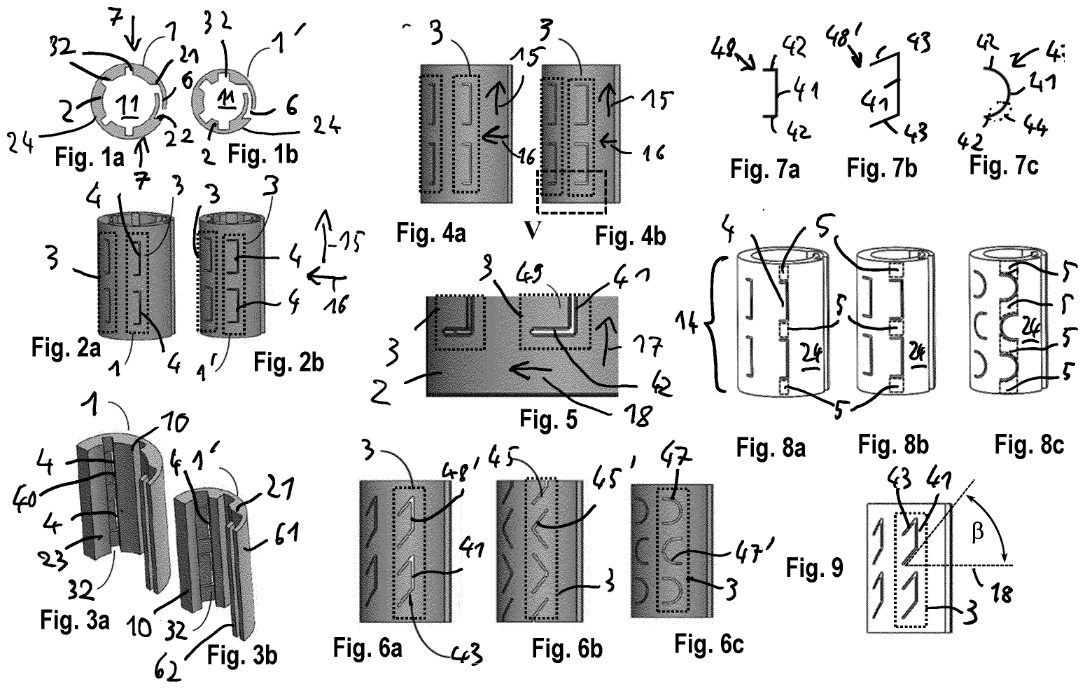

As illustrated by the first and second embodiment depicted in FIGS. 1 to 5, the augment 1 is also available in different sizes, for example also as a smaller sized augment 1′.

As specifically illustrated in FIG. 2a, b the wall 2 comprises at least one, preferably a plurality of living hinges 3, each comprising at least one, preferably a plurality of bent slits 4. In addition to said bent slit(s) 4, the living hinge 3 may further comprise a grooving 32 which in the depicted embodiments is located on its interior face (towards the channel 11) of the wall 2. The living hinge(s) 3 allow for a substantially increased elasticity of the augment 1, 1′ under a compressional force 7 which is exerted on an exterior face of the wall 2. In the depicted embodiment, each of the living hinges 3 comprises a plurality of bent slits 4 which in the depicted embodiment are configured as being U-shaped; however, one of such bent slits 4 per living hinge 3 can be sufficient. Likewise, having only one living hinge 3 can be sufficient, too. However, if plural living hinges 3 are provided then they are arranged sequentially along a circumferential direction 16 of the wall 2. The circumferential direction 16 is essentially perpendicular to the longitudinal direction 15.

For further definitions of the directional terms used here and in the following, reference is made to the explanation given above.

As it specifically can be seen in FIG. 3a, b, the bent slits 4 are through openings reaching through the entire thickness of the respective portion of the wall 2, which means that a depth 40 of the bent slits 4 is the same as the thickness of the wall 2 at the location of the respective bent slit 4.

As further can be specifically seen in FIG. 1a, b and FIG. 3a, b, the wall 2 further comprises a compensator element 6 which is formed by a longitudinal wall void 60 which separates the wall 2 such that it is non-contiguous and two edges 21 and 22 are formed, one at either side of the wall void 60.

Details of the configuration of the bent slits 4 of the living hinge 3 are shown in FIG. 4a, b and in a blow-up section as presented in FIG. 5. Each of the depicted bent slits 4 comprises a vertical section 41 which acts as a base portion and, at either end thereof, a horizontal section 42. Thereby U-shape 48 is formed. The portion within the “U” which is encircled at 3 sides by horizontal and vertical portions 41, 42 of the bent slit 4 is termed to be a flap 49. The horizontal portion 42 ensures that a considerable amount of area would be created forming the flap 49. The material of the flap 49 is “passive” in that it does not contribute to elastic forces. By virtue of this, the importance of the horizontal extension of the bent slits 4 for increasing and adjusting elasticity can be appreciated.

As it can be further appreciated, each of the living hinges 3 may comprise a plurality of the bent slits 4. To this end, the bent slits 4 are being arranged in a series, i.e. one next to the other in a vertical direction 17 which is orthogonal to the circumferential direction 16 of the wall 2. Strips of solid material will remain between the stacked bent slits by stacking one bent slit 4 on top of the other thereby producing a series, and thereby—for a comparable elasticity of the resulting living hinge 3—an increase of the stability of the cone augment 1 can be achieved as opposed to having a living hinge with just a single long slit.

The “U”-shape 48 is an example of realizing a horizontal extension. The invention has realized that this extension does not need to be exactly horizontal, it only needs to have a significant horizontal component. This can also be achieved by an oblique section 43 of the bent slit 4, thereby forming a skewed U-shape 48′, as depicted in FIG. 6a showing a third embodiment. On the same token, two (or more) oblique sections 43 can be combined to form a shape like a chevron 45, as depicted in FIG. 6b. Such a shape has the benefit of providing the necessary extension in vertical as well as in horizontal direction 17, 18. Such a chevron like bent slit 4 is efficient in terms of being manufactured and providing elasticity to the living hinge 3.

The chevron shape 45 as depicted is rather sharp angled at the junction of the two oblique sections 43. This, however, is not a necessity: a transition 44 can also be rounded, be it a rounded tip or surrounding being a large one essentially forming an arc which could be a semi-circular arch 47, as depicted in FIG. 6c. Such a curved configuration features the benefit of providing curvature instead of sharp bends, thereby decreasing the risk that may arise due to a notch effect.

Schematic drawings of some examples of basic configurations of the bent slits 4 are provided in FIG. 7a, b, c. The ordinary U-shaped form 48 is shown in FIG. 7a, the skewed-U shape 48′ in FIG. 7b, and the curved configuration of, e.g. as a semi-circular arch 47, is shown in FIG. 7c. Indications of effective vertical and horizontal portions 41, 42 as well as of transitions 44 are also provided in these figures.

It is to be noted that a series of bent slits forming the living hinge 3 can employ the individual bent slits 4 being sequentially arranged in a position, like bent slits 4 being arranged in a series of chevron 45 and rotated chevron 45′, preferably rotated by 180°, as depicted in FIG. 6b. Likewise the, this could be applied to the rounded semi-circular arch 47 being in series with a rotated semi-circular arch 47′, also preferably rotated by 180°, as depicted in FIG. 6c.

Between the two neighbouring bent slits 4 or between a peripheral bent slit 4 its respective neighbouring top or bottom of the wall 2, bending strips 5 are formed. These bending strips 5 are those portions of material where the wall 2 is continuous at the living hinge 3, i.e. which portions of the wall 2 do effectively contribute to elasticity. Under compressional load 7, those portions are creating the counter-force which is required for elasticity. Those portions are marked by a dashed rectangle in FIGS. 8a, b and a more complex shape in FIG. 8c. As it can be readily appreciated by comparing FIG. 8b to FIG. 8a, in FIG. 8b the horizontal extension of the bent slits 4 is longer producing bending strips 5 which have the same width but a bigger length than that of FIG. 8a. Thus, the bending strips of FIG. 8b are longer and therefore softer, Like a longer leaf spring is softer than a short one. In effect, the augment of FIG. 8b features a bigger elasticity than the augment of FIG. 8a, and the degree of elasticity can be easily adjusted by dimensioning of the horizontal and vertical dimensions of the bent slits 4.

In order to further ease of manufacturing, it is preferred to employ additive manufacturing methods, as already mentioned above, wherein it is presupposed that the build direction of the manufacturing process is in vertical direction. In order to effectively manufacture horizontal structures, in particular those portions of the bent slit 4 extending horizontally, a skewed configuration is beneficial. Preferably a skewing angle β is selected such as to be at least 45° to the horizontal direction, preferably 50° to 65°, further preferably less than 70°, yet further about 55° as depicted in FIG. 9. This ensures efficient manufacturing by said additive manufacturing methods and obviates any difficulties that may arise if exactly horizontal structures are to be produced without any additional support.

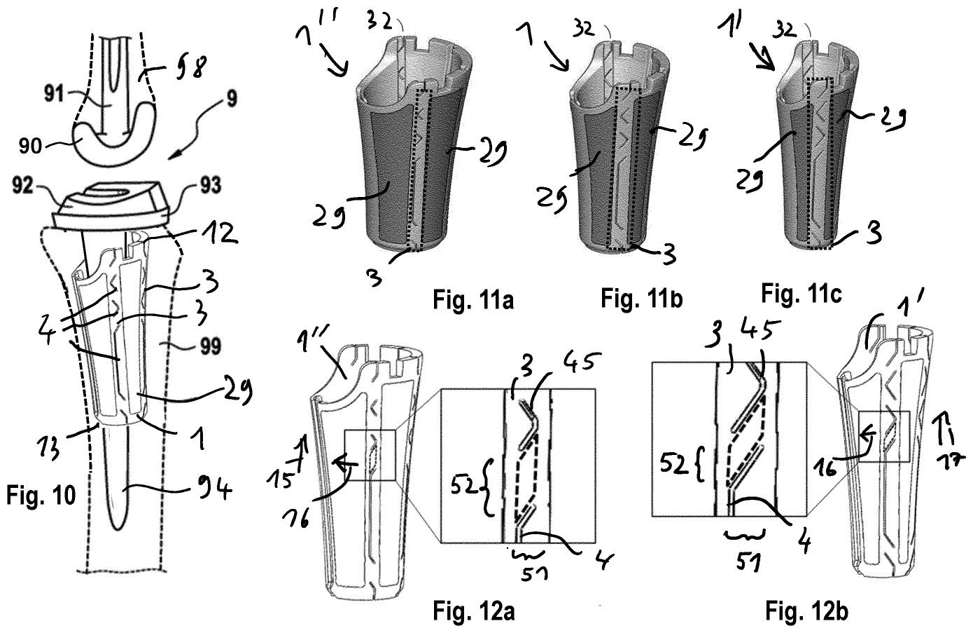

An example of an embodiment of an augment placed in situ at a large bone is depicted in FIG. 10. It is shown to be implanted into a proximal portion of a tibia bone 99. The augment 1 is placed into a cavity of an upper portion of the tibia 99, thereby forming a base on which a tibial plate 93 of a tibial component 92 of a knee prosthesis 9 is to be positioned. The tibial component 92 comprises a stem 94 configured to be anchored in a medullary channel of the tibia bone 99. The stem 94 extends through the channel 11 of the augment device 1.—Said knee endoprosthesis 9 further comprises a femoral component 90 configured for rotatable interaction with the tibial portion 92. Said femoral component 90 comprises a stem 91 to be placed into the distal end of a femoral bone 98. Likewise, a similar augment device (not shown) Like the depicted augment device can be provided at said distal end of the femoral bone 98.

Typically, a fixation of the stem 91 (or 94) in the channel 11 will be achieved by means of bone cement (not shown). For this reason, an inner face 23 of the wall 2 forming the hollow body 10 is made to have a solid surface. Conversely, an outer face 24 of the wall 2 is to be fixated to the surrounding bone in a cementless manner. To this end, the porous structure 29 is provided at the outer face 24 of the wall 2, preferably within pockets provided at said outer face 24, these pockets being filled with the porous structure 29. Said porous structure 29 is configured to promote ingrowth of bony tissue. To this end, the porous structure is configured to provide at least one, preferably multiple layers of pores, with pores ranging between 0.3 and 1.5 mm in width, preferably between 0.5 and 1 mm. Thereby, a stable long-term fixation of the medical implant into the bone can be achieved.

A sixth embodiment comprising a set of cone augments having different sizes shown in FIG. 11a, b, c. The cone augment 1 having a medium-size is shown in FIG. 11b, whereas essentially the same cone augment just having a larger size 1″ is shown in FIG. 11a, and another essentially the same augment just having a smaller size 1′ is shown in FIG. 11c. It is to be noted, that owing to the varied dimensions of the living hinge 3 with its bent slits 4 elasticity can be adjusted, in the depicted case it is adjusted so as to be identical for all the differently sized cone augments 1, 1′, 1″. Having the possibility of such identical elasticity is a huge benefit for the surgeon it. It is the smallest cone augment 1′ that requires the largest dimensioned slits, and the invention provides by its bent slits 4 the means for achieving living hinges 3 with the required increased elasticity. In addition, the invention allows providing bent slits 4 having an even bigger horizontal extension, thereby achieving a further increased elasticity, which may be beneficial particularly for the smaller augment 1′.

This is depicted in more detail in FIG. 12a, b showing a seventh embodiment which is identical to the sixth embodiment except does not feature grooves 32. Compared are the large-sized augment 1″ with an additional blow-up of the configuration of the bending strip 5 in FIG. 12a, and the small augment 1′ with an additional blow-up of the configuration of its bending strip 5 is shown in FIG. 12b.

As it can be readily appreciated, the horizontal extension of the bent slit 4 as measured by a length 51 of the bending strip 5 (seen in the circumferential direction 16) is larger in the case of the bent slit 4 of the smaller augment 1′ shown in FIG. 12b in contrast to that of the large-sized augment 1″ shown in FIG. 12a. Moreover, in the case of the smaller sized augment 1′ the bent slits 4 are more closely spaced resulting in bending strips 5 having a narrower width 52 (seen in vertical direction 17) as opposed to the bent slits 4 of the large-sized augment 1″. Both differences contribute to an increased length/width ratio of the bending strip 5 of the smaller sized augment 1′, thereby allowing adjustment of elasticity such that it becomes equal between various sizes.

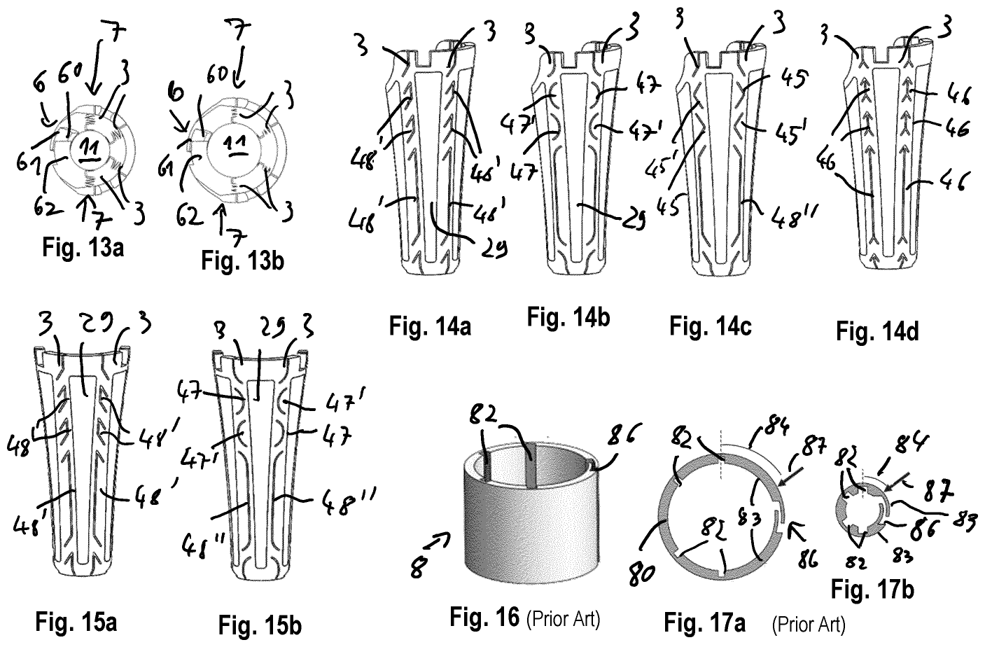

A top view of the seventh embodiment is provided FIG. 13a, b. Accordingly, the relationship of the several living hinges 3 being placed essentially equidistantially around the circumference of the wall 2. Further, the compensator element 6 with his wall void 60 and tongue-like extension 61, 62 at the respective edges 21, 22 of the wall 2 can be seen. The tongue-like extension 61, 62 are dimensioned such to overlap each other regardless of a compression state of the cone augment 1, 1′ under the load of the compressional force 7. Thereby, the wall void 60 is always covered by the extension 61, 62 which are configured such as to only leave a small slit having a width of approximately 0.5 mm, thereby forming an effective cement seal against unwanted outflow of bone cement placed in the channel 11 towards the exterior of the hollow body 10, in particular into the porous structure that may be present at the outer face 24 of the wall 2.

The bent slits 4 may take different configurations, as already indicated. In FIG. 14a-d lateral views of cone augments 1 having different embodiments of series of bent slits 4 are shown, and in FIG. 15a, b frontal views corresponding to the embodiment shown in FIG. 14a, b are shown. The bent slits 4 may take a skewed-U 48′ shape, a curved configuration comprising arches 47, a combination of chevrons 45 with an oppositely skewed U-configuration 48″, and a configuration comprising the bent slits configured as an arrow 46, having oblique portions pointing in opposite directions at either end of the vertical section thereby forming structure similar to a tip as well as rear feathers of the arrow 46. By varying configuration, number as well as length of the individual bent slits 4 elasticity of the living hinge 3 can be adjusted.

FIG. 16 shows an augment 8 according to the prior art having a compensator element 86 as well as separate bending joints, each formed by a simple linear longitudinal groove 82 the wall 80. The difficulty encountered thereby is that in the prior art different sizes, as depicted in FIG. 17a, b, provide wall segments 83 being much longer for the larger sized augment than for the smaller sized. This leads to different effective lever length 84 and results in a situation wherein the same compressive force 87 results in lesser bending effect, thereby putting a bigger strain on smaller bones. This can have adverse effects on the integrity of the bone and health of the patient. This drawback of the prior is overcome by the invention, as explained above.

1. A medical implant for a joint endo-prosthesis, comprising:
a sleeve-shaped hollow body having a wall surrounding a channel in a circumferential direction,
wherein the channel extends in a longitudinal direction from a bottom to a top of the hollow body, the wall comprising at least one living hinge oriented in the longitudinal direction, said living hinge being configured for compressing the channel, the living hinge being formed by at least one elongated surface opening,
wherein
said elongated surface opening is a bent slit that extends as a slit at least in a vertical and in a horizontal direction.

2. The medical implant of claim 1, wherein the bent slit is curved and/or angled.

3. The medical implant of claim 1, wherein at least said extending in the horizontal direction is formed by a section of the bent slit having an oblique orientation.

4. The medical implant of claim 1, wherein an oblique section is oriented at an angle (β) of at least 45°.

5. The medical implant of claim 1, wherein the bent slit has a depth of at least a third of a thickness of wall at a location of the living hinge.

6. The medical implant of claim 1, wherein the bent slit is configured as a through opening.

7. The medical implant of claim 1, wherein a groove is provided for the living hinge, said groove configured in a longitudinal direction at an inner and/or at an outer face of the wall.

8. The medical implant of claim 1, wherein the bent slit has a width configured to block passage of bone cement.

9. The medical implant of claim 1, wherein the bent slit is formed as a chevron having at least two sections being oriented obliquely to the vertical direction.

10. The medical implant of claim 1, wherein the bent slit is formed by a vertical section and at least one horizontal section positioned at an end portion of the vertical section, said at least one horizontal section being oriented oblique or orthogonal to the vertical section.

11. The medical implant of claim 1, wherein the bent slit is formed to have a shape like a feathered arrow.

12. The medical implant of claim 1, wherein a transition between sections is sharp angled or curved.

13. The medical implant of claim 1, wherein the bent slit is continuously curved.

14. The medical implant of claim 1, wherein a bending strip is formed between the bent slit and a respective adjacent top and/or bottom of the wall, and between each two neighboring bent slits of the bent slits forming the living hinge.

15. The medical implant of claim 1, wherein a width of a bending strip is dimensioned such as to achieve a predetermined elasticity of the living hinge.

16. The medical implant of claim 10, wherein a plurality of oblique bending strips are formed between three or more of the bent slits having oblique sections, said bending strips being oriented obliquely in opposite directions.

17. The medical implant of claim 1, wherein the hollow body comprises a compensator element configured for adjusting a circumference of the wall in a compressed state.

18. The medical implant of claim 17, wherein the compensator element is configured as a longitudinal wall void extending from the bottom to the top of the wall to form a first and second edge of the wall separated by a width of said longitudinal wall void, said edges being provided with overlapping tongue-like extensions arranged in a sliding relationship.

19. The medical implant of claim 1, wherein a plurality of the living hinges are provided along the wall spaced equidistant and/or equiangular along a circumference of the wall.

20. The medical implant of claim 1, wherein the wall has an inner face toward the channel and an outer face on an opposing side of the wall, the outer face at least partially comprises a porous structure configured for bone ingrowth, wherein the inner face is solid.

21. A set of medical implants of claim 1, wherein the set comprises a plurality of such medical implants comprising at least one larger medical implant (1″) and one smaller medical implant (1′).

22. The set of medical implants according to claim 21, wherein the smaller medical implant (1′) features a bending strip having a higher length/width ratio than that of the bending strip of the larger medical implant (1″).
